SEQUENCE LISTING (1 sequence)

SEQ ID NO: 1 - DNA, 600 bp, Candida palmioleophila
  source 1..600: mol_type = other DNA
tatgcttaagttcagcgggtaatcctacctgatttgaggtcaaacttgtttgttgttttttaaggcaaagcctaacaccaaaaattcacaaccaagaattgtcaacgagttggataaacctaatacattgatatttcctgaatgcacttctcagcgtcactcatgccaataaatttcaagcaaacgcctagttcgactaagagtatcactcaataccaaacccaagggtttgagagagaaatgacgctcaaacaggcatgccctctggaataccagagggcgcaatgtgcgttcaaagattcgatgattcacgaaaatctgcaattcatattacttatcgcatttcgctgcgttcttcatcgatgcgagaaccaagagatccgttgttgaaagttttgaagattgtttgaatttaatcaacaaattgacaaagtattaaataacaattcaattaaaaattgaagtttgtgtaaacctctggcccgactaatttcttagccaagccaaagcaatagtttctaataaagaaaaacattgtgtgtaaggtttaatcgccgcgcaattaagcgctggcaattagaatacagtaatgatccttcc

(19) United States
(12) Patent Application Publication
(10) Pub. No.: US 2026/0008992 A1
(43) Pub. Date: Jan. 8, 2026

(54) STRAIN OF CANDIDA AND APPLICATION THEREOF

(71) Applicant: XIANGHU LABORATORY, Zhejiang (CN)

(72) Inventors: Gang ZHENG, Zhejiang (CN); Yan LIU, Zhejiang (CN); Yanjie LI, Zhejiang (CN); Baolei JIA, Zhejiang (CN); Yanlai YAO, Zhejiang (CN)

(73) Assignee: XIANGHU LABORATORY, Zhejiang (CN)

(21) Appl. No.: 19/103,827

(22) PCT Filed: Apr. 12, 2024
(86) PCT No.: PCT/CN2024/087444

§ 371 Date: Feb. 14, 2025

(30) Foreign Application Priority Data
Mar. 4, 2024 (CN) 202410241003.8
Mar. 11, 2024 (CN) 202410273160.7

(51) Int. Cl. C12N 1/16; B09C 1/10; C12R 1/72

(52) U.S. Cl. CPC C12N 1/165; B09C 1/10; C12R 2001/72

(57) ABSTRACT

The present invention relates to the field of microorganisms, and discloses a strain of Candida and application thereof. The strain of Candida has a name of XHZG06-95A3, belongs to Candida palmioleophila in taxonomy, and has a preservation number of CGMCC No. 29215. The C. XHZG06-95A3 provided by the present invention has the capacity of increasing ambient pH and dissolving P components, can reduce the acidity of acidified soil and increase the available phosphorus content therein, and can adapt to a wide range of ambient temperatures and pH values. This is the first time that a strain capable of increasing ambient pH and dissolving P components has been found in the species of C. palmioleophila.

### TECHNICAL FIELD

The present invention relates to the field of microorganisms, and in particular to, a strain of Candida XHZG06-95A3 and application thereof in improving acidified soil.

### RELATED ART

The pH value of soil is determined by a chemical balance between acidic and alkaline substances in the soil. In a case where such a chemical balance is destroyed, excessive acidic substances and insufficient alkaline substances in the soil will cause soil acidification. In recent years, the problem of soil acidification has become increasingly severe due to the excessive use of acidic fertilizers and the influence of human activities such as continuous cropping, posing a growing threat to the ecological environment and agricultural production. Acidification will increase the activity of heavy metal elements in the soil, reduce soil fertility, and decrease the absorption rate of nutrient elements. For example, in acidified soil, phosphate ions are easily bound to iron and aluminum ions and fixed, resulting in their loss of fertilizer efficiency and difficulty in being absorbed and utilized by plants.

By applying soil improvement agents, soil acidification can be alleviated. Currently used acidified soil improvement agents are mainly chemical improvement agents such as lime, which pose hidden dangers of soil hardening and fertility decline. In comparison, microbial improvement agents have less adverse effects on soil. Therefore, in recent years, the development of methods and related strain resources for microbial improvement of acidified soil has received increasing attention. Acidified soil improvement bacteria are required to have good stress resistance, be able to adapt to acidified soil environments, and be capable of utilizing acidic substances in the soil or secreting alkaline substances. At present, due to the limitations of strain resources as well as the stress resistance of microorganisms and their adaptability to acidified soil, how to improve the remediation effect of microbial improvement agents on acidic soil remains a core issue of concern to researchers.

Candida palmioleophila is a species in Candida, and currently there is no unified Chinese translation for this species. At present, research on this species mainly focuses on the field of wastewater treatment. It has been found that some strains (such as a strain WW7 disclosed in patent CN112961790B) in this species have the function of removing ammonia nitrogen, TN and COD in wastewater, or that some strains (such as a strain NOB-1 disclosed in patent CN114292762B) in this species have heterotrophic nitrification and aerobic denitrification functions, but it has not been reported that any strain in this species has the function of improving acidified soil.

### SUMMARY OF INVENTION

In order to solve the technical problems of limited strain resources for improvement of acidified soil and poor adaptability of strains to acidified soil, the present invention provides a C. XHZG06-95A3. The C. XHZG06-95A3 has the capacity of increasing ambient pH and dissolving P components, can reduce the acidity of acidified soil and increase the available phosphorus content therein, and can adapt to a wide range of ambient temperatures and pH values.

The present invention provides a method for preparing an acidified soil improvement agent based on microbial fermentation. The acidified soil improvement agent prepared by the method can quickly increase the pH of acidified soil, while increasing the soluble phosphorus content in the soil, thus having a good effect on the acidified soil remediation.

The specific technical solutions of the present invention are as follows:

In a first aspect, the present invention provides a strain of C. XHZG06-95A3, which belongs to C. palmioleophila in taxonomy, with a preservation number of CGMCC No. 29215.

The C. XHZG06-95A3 provided by the present invention belongs to C. palmioleophila (which currently does not have a unified Chinese translation). It was preserved in the China General Microbiological Culture Collection Center (CGMCC) on Dec. 4, 2023, with the address of CGMCC being No. 3, 1 #Courtyard, Beichen West Road, Chaoyang District, Beijing.

The strain provided by the present invention has the capacity of increasing ambient pH, and its mechanism may be achieved by using acidic substances in the environment to reduce the acidity of the environment, and by producing amine substances to neutralize hydrogen ions in the environment. Moreover, the strain also has the capacity of dissolving P components, which can transform insoluble phosphorus into soluble phosphorus. Under the above effects, the strain provided by the present invention can be used for improving acidified soil. When applied to acidified soil, it can reduce soil acidity and increase the available phosphorus content that can be absorbed and utilized by plants in the soil.

In addition, the strain provided by the present invention has a high adaptability to different ambient pH values and temperatures. The experimental results showed that the strain could grow well at the ambient pH of 3-8 and the ambient temperature of 15-35° C., while effectively increasing the ambient pH value; and the strain could effectively play a role in dissolving P components in the environments with pH values of 4-7 and temperatures of 15-35° C.

In a second aspect, the present invention provides a method for preparing an acidified soil improvement agent based on microbial fermentation, including the following steps:
(1) inoculating an acidified soil improvement bacterium into a fermentation culture medium for first fermentation to obtain a primary fermentation broth, where the acidified soil improvement bacterium is the C. XHZG06-95A3 belonging to C. palmioleophila in taxonomy, and having a preservation number of CGMCC No. 29215;
   (2) adding mussel shell powder to the primary fermentation broth for second fermentation to obtain a secondary fermentation broth; and
   (3) separating products from the secondary fermentation broth to obtain an acidified soil improvement agent.

According to the present invention, the acidified soil improvement bacterium (C. XHZG06-95A3) with the function of dissolving P components is used for the first fermentation, which can enable rapid strain amplification and secretion of substances capable of increasing soil pH. Then, the mussel shell powder is added for the second fermentation, and the insoluble phosphorus in the mussel shell powder can be transformed into soluble phosphorus by the acidified soil improvement bacterium with the function of dissolving P components, so that the acidified soil improvement agent provided by the present invention can increase the content of soluble phosphorus in acidified soil to a greater extent; and moreover, the mussel shell powder contains calcium carbonate and calcium oxide, which can neutralize soil acidity, thereby improving the improvement effect of acidified soil. When the acidified soil improvement agent prepared by the present invention is applied to acidified soil, the acidified soil improvement bacterium in the acidified soil improvement agent, the substances capable of increasing soil pH which are produced during the first fermentation of the acidified soil improvement bacterium, and the mussel shell powder can be utilized to quickly increase soil pH. Furthermore, the acidified soil improvement bacterium can also be used for transforming insoluble phosphorus in the soil into soluble phosphorus, and the soluble phosphorus released from the mussel shell powder during the second fermentation process can be utilized, so that the available phosphorus content in the soil is quickly increased.

Preferably, in step (1), the fermentation culture medium is prepared from the components at the following concentrations: 5-10 g/L yeast extract, 10-20 g/L glucose, 10-20 g/L peptone, 0.5-1 g/L K₂HPO₄, 3-6 g/L (NH₄)₂HPO₄, 0.5-1 g/L Mg₂SO₄·7H₂O, and 0-0.3 g/L KCI, with water used as a solvent and pH being 6-7.

Preferably, in step (1), the primary fermentation broth has an OD₆₀₀ of 0.7-1.2.

Preferably, in step (2), a mass-to-volume ratio of the mussel shell powder to the first fermentation broth is 2-8 g:100 mL.

Preferably, in step (2), a carbon source is added to the primary fermentation broth before the second fermentation is carried out, where the carbon source includes one or more of glucose, galactose, sucrose, and starch.

By adding a carbon source, the strain XHZG06-95A3 can be promoted to play a role in dissolving P components during the second fermentation, allowing insoluble phosphorus in mussel shell powder to be transformed into soluble phosphorus that can be absorbed and utilized by plants. Moreover, for the strain XHZG06-95A3 used in the present invention, its function of dissolving P components is further promoted when using one or more of glucose, galactose, sucrose, and starch as the carbon source.

Further, a mass-to-volume ratio of the carbon source to the primary fermentation broth is 0.5-2.0 g:100 mL.

Preferably, in step (2), before the mussel shell powder is added to the primary fermentation broth, the mussel shell powder is calcined at 550-650° C. for 3-4 h.

By calcining the mussel shell powder, the organic matter in the mussel shell powder is decomposed, and the generated gas escapes at the same time, which can increase the porosity of the mussel shell powder and expand the pore capacity, thus being conducive to the adsorption and reaction of acidic substances in the soil by the mussel shell powder, and improving the effect of acidified soil improvement.

Preferably, in step (1), the first fermentation is carried out at 20-30° C.

Preferably, in step (2), the second fermentation is carried out at 20-30° C. for 1-5 days.

Preferably, in step (1), the specific process of inoculating the acidified soil improvement bacterium into the fermentation culture medium includes the following steps: inoculating the acidified soil improvement bacterium into an activation medium for activation, and then picking single colonies from the activation medium and inoculating the same into the fermentation culture medium.

Further, the activation medium is an LB solid medium.

Preferably, in step (2), the mussel shell powder has a particle size of less than or equal to 10 μm.

In a third aspect, the present invention provides an acidified soil improvement agent prepared by using the method.

In a fourth aspect, the present invention provides application of the C. XHZG06-95A3 or the acidified soil improvement agent in improving acidified soil.

Compared with the prior art, the present invention has the following advantages:
(1) The C. XHZG06-95A3 provided by the present invention has the capacity of increasing ambient pH and dissolving P components, can reduce the acidity of acidified soil and increase the available phosphorus content therein, and can adapt to a wide range of ambient temperatures and pH values. This is the first time that a strain capable of increasing ambient pH and dissolving P components has been found in the species of C. palmioleophila.
(2) The present invention adopts the C. XHZG06-95A3 with the function of dissolving P components, combined with the first fermentation and the second fermentation process performed after adding mussel shell powder, and thus the prepared acidified soil improvement agent can quickly increase the pH of the acidified soil, and increase the soluble phosphorus content in the soil.

### BRIEF DESCRIPTION OF DRAWINGS

FIG. 1 shows the effect of a strain XHZG06-95A3 on the improvement of acidified soil.

FIG. 2 is a photograph of a colony of the strain XHZG06-95A3.

FIG. 3 shows the effect of an initial pH on the growth of the strain XHZG06-95A3.

FIG. 4 shows the effect of the initial pH on the pH raising effect of the strain XHZG06-95A3.

FIG. 5 shows the effect of temperature on the growth of the strain XHZG06-95A3.

FIG. 6 shows the effect of the temperature on the pH raising effect of the strain XHZG06-95A3.

FIG. 7 shows the effect of the temperature on the capacity of dissolving P components by the strain XHZG06-95A3.

FIG. 8 shows the effect of the initial pH on the capacity of dissolving P components by

the strain XHZG06-95A3.

FIG. 9 shows the changes in soluble phosphorus concentration in a fermentation broth during the second fermentation process.

### DESCRIPTION OF EMBODIMENTS

The present invention will be further described with reference to the following examples. It should be understood that these examples are only used for illustrating the present invention and are not intended to limit the scope of the present invention. Any changes and advantages that can be conceived by those skilled in the art without departing from the spirit and scope of the inventive concept shall be included in the present invention, and the appended claims and any equivalents thereof are all within the scope of protection of the present invention.

The “strain XHZG06-95A3” mentioned in the following examples refers to the strain that was preserved in the China General Microbiological Culture Collection Center (CGMCC) on Dec. 4, 2023, with the preservation number being CGMCC No. 29215. Its microbial classification was named C. palmioleophila.

### EXAMPLE 1

### Isolation and Identification of Strain XHZG06-95A3

The strain XHZG06-95A3 was isolated from coastal soil. Its morphological, physiological and biochemical characteristics, as well as its 16S rDNA sequence are as follows:

### (1) Morphological and Physiological Biochemical Characteristics:

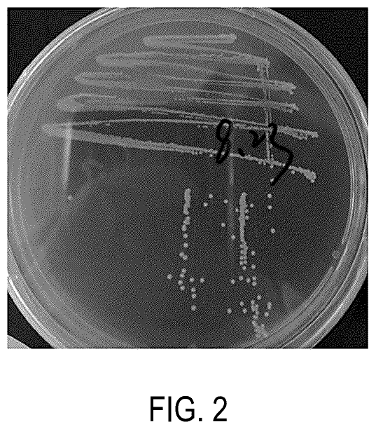

The colony morphology of the strain XHZG06-95A3 is shown in FIG. 2.

(2) 16S rDNA Sequence:

The 16S rDNA sequence of the strain XHZG06-95A3 has a length of 600 bp, which is as shown in SEQ ID NO: 1, specifically as follows:

| | |
|---|---|
| tatgcttaag ttcagcgggt aatcctacct gatttgaggt caaacttgtt tgttgttttt | 60 |
| taaggcaaag cctaacacca aaaattcaca accaagaatt gtcaacgagt tggataaacc | 120 |
| taatacattg atatttcctg aatgcactte tcagcgtcac tcatgccaat aaatttcaag | 180 |
| caaacgccta gttcgactaa gagtatcact caataccaaa cccaagggtt tgagagagaa | 240 |
| atgacgctca aacaggcatg ccctctggaa taccagaggg cgcaatgtgc gttcaaagat | 300 |
| tcgatgattc acgaaaatct gcaattcata ttacttateg catttcgctg cgttcttcat | 360 |
| cgatgcgaga accaagagat ccgttgttga aagttttgaa gattgtttga atttaatcaa | 420 |
| caaattgaca aagtattaaa taacaattca attaaaaatt gaagtttgtg taaacctctg | 480 |
| gcccgactaa tttcttagcc aagccaaagc aatagtttct aataaagaaa aacattgtgt | 540 |
| gtaaggttta atcgccgcgc aattaagcgc tggcaattag aatacagtaa tgatccttcc. | 600 |

After identification, the strain XHZG06-95A3 belongs to C. palmioleophila (there is currently no unified Chinese translation for this species).

### EXAMPLE 2

### Strain XHZG06-95A3′s pH Tolerance and Ability to Reduce Environmental acidity

In order to explore the adaptability of a strain XHZG06-95A3 to different pH environments, this example tested the growth of the strain XHZG06-95A3 and the ability thereof to increase ambient pH at different initial pH values. The specific process and test results are as follows:

### 2.1 Preparation of Medium

An LB solid medium and a culture medium were prepared according to the following formulas.

### (1) LB Solid Medium:

10 g of peptone, 5 g of yeast extract, 10 g of NaCl and 15 g of agar were dissolved in 950 mL of distilled water, and then distilled water was added into the obtained solution to make up to 1 L; the pH was adjusted to 6.0 with dilute sulfuric acid and a sodium hydroxide solution; and the product was sterilized with high-pressure steam at 121° C. for 20 min.

### (2) Culture Medium:

10 g/L yeast extract, 15 g/L glucose, 20 g/L peptone, 0.5 g/L K₂HPO₄, 5 g/L (NH₄)₂HPO₄, 0.5 g/L Mg₂SO₄·7H₂O and 0.1 g/L KCl were dissolved in 950 mL of distilled water, and then distilled water was added into the obtained solution to make up to 1 L; the pH was adjusted to a desired value with dilute sulfuric acid and a sodium hydroxide solution; and the product was sterilized with high-pressure steam at 121° C. for 20 min.

### 2.2 Effect of Initial pH Values on Growth of Strain XHZG06-95A3 and Ability Thereof to Increase Ambient pH

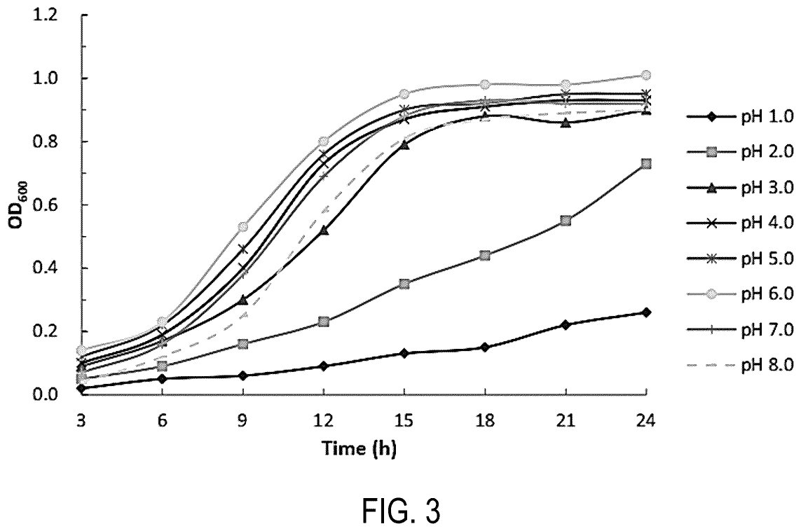

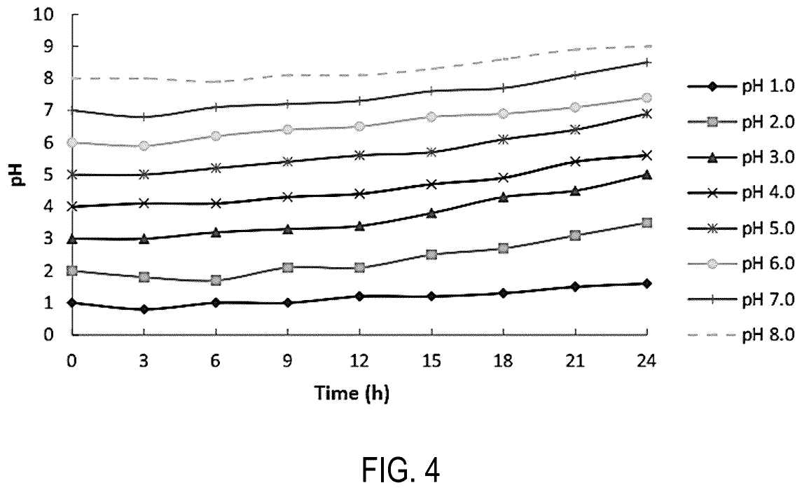

The strain XHZG06-95A3 was inoculated into an LB solid medium, and cultured at 25° C. until single colonies grew out; the single colonies were picked and respectively inoculated into culture media with pH of 1.0, 2.0, 3.0, 4.0, 5.0, 6.0, 7.0 and 8.0; shake culture was carried out at 25° C. and 180 r/min, and samples were taken at regular intervals; and the absorbance values OD₆₀₀ of the culture media at 600 nm and the pH values thereof were detected. Table 1, Table 2, FIG. 3 and FIG. 4 show the changes in OD₆₀₀ and pH values of the culture media over time at different initial pH values.

TABLE 1
Effect of initial pH values on growth of strain XHZG06-95A3

| Initial | OD₆₀₀ values of a culture medium after different time | | | | | | | |
|---|---|---|---|---|---|---|---|---|
| pH | 3 h | 6 h | 9 h | 12 h | 15 h | 18 h | 21 h | 24 h |
| pH 1.0 | 0.02 | 0.05 | 0.06 | 0.09 | 0.13 | 0.15 | 0.22 | 0.26 |
| pH 2.0 | 0.05 | 0.09 | 0.16 | 0.23 | 0.35 | 0.44 | 0.55 | 0.73 |
| pH 3.0 | 0.09 | 0.17 | 0.30 | 0.52 | 0.79 | 0.88 | 0.86 | 0.90 |
| pH 4.0 | 0.10 | 0.19 | 0.40 | 0.73 | 0.87 | 0.91 | 0.93 | 0.93 |
| pH 5.0 | 0.12 | 0.22 | 0.46 | 0.76 | 0.90 | 0.92 | 0.95 | 0.95 |
| pH 6.0 | 0.14 | 0.23 | 0.53 | 0.80 | 0.95 | 0.98 | 0.98 | 1.01 |
| pH 7.0 | 0.07 | 0.16 | 0.38 | 0.69 | 0.88 | 0.93 | 0.92 | 0.92 |
| pH 8.0 | 0.04 | 0.12 | 0.25 | 0.58 | 0.81 | 0.87 | 0.89 | 0.90 |

TABLE 2
Effect of initial pH values on pH raising
effect of strain XHZG06-95A3

| Initial | pH values of a culture medium after different time | | | | | | | | |
|---|---|---|---|---|---|---|---|---|---|
| pH | 0 h | 3 h | 6 h | 9 h | 12 h | 15 h | 18 h | 21 h | 24 h |
| pH 1.0 | 1.0 | 0.8 | 1.0 | 1.0 | 1.2 | 1.2 | 1.3 | 1.5 | 1.6 |
| pH 2.0 | 2.0 | 1.8 | 1.7 | 2.1 | 2.1 | 2.5 | 2.7 | 3.1 | 3.5 |
| pH 3.0 | 3.0 | 3.0 | 3.2 | 3.3 | 3.4 | 3.8 | 4.3 | 4.5 | 5.0 |
| pH 4.0 | 4.0 | 4.1 | 4.1 | 4.3 | 4.4 | 4.7 | 4.9 | 5.4 | 5.6 |
| pH 5.0 | 5.0 | 5.0 | 5.2 | 5.4 | 5.6 | 5.7 | 6.1 | 6.4 | 6.9 |
| pH 6.0 | 6.0 | 5.9 | 6.2 | 6.4 | 6.5 | 6.8 | 6.9 | 7.1 | 7.4 |
| pH 7.0 | 7.0 | 6.8 | 7.1 | 7.2 | 7.3 | 7.6 | 7.7 | 8.1 | 8.5 |
| pH 8.0 | 8.0 | 8.0 | 7.9 | 8.1 | 8.1 | 8.3 | 8.6 | 8.9 | 9.0 |

Analysis and conclusion of the test results: It can be seen from Table 1, Table 2, FIG. 3 and FIG. 4 that the strain XHZG06-95A3 has good acid resistance and can survive at pH of 1 to 8, where it can grow well in an environment with pH of 3 to 8 and can also effectively increase the pH of the culture media.

### EXAMPLE 3

### Strain XHZG06-95A3's Temperature Tolerance and Ability to Reduce Environmental Acidity

In order to explore the adaptability of a strain XHZG06-95A3 to different temperature environments, this example tested the growth of the strain XHZG06-95A3 and the ability thereof to increase ambient pH at different temperatures. The specific process and test results are as follows:

### 3.1 Preparation of Medium

An LB solid medium and a culture medium were prepared according to the following formulas.

### (1) LB Solid Medium:

10 g of peptone, 5 g of yeast extract, 10 g of NaCl and 15 g of agar were dissolved in 950 mL of distilled water, and then distilled water was added into the obtained solution to make up to 1 L; the pH was adjusted to 6.0 with dilute sulfuric acid and a sodium hydroxide solution; and the product was sterilized with high-pressure steam at 121° C. for 20 min.

### (2) Culture Medium:

10 g/L yeast extract, 15 g/L glucose, 20 g/L peptone, 0.5 g/L K₂HPO₄, 5 g/L (NH₄)₂HPO₄, 0.5 g/L Mg₂SO₄·7H₂O and 0.1 g/L KCI were dissolved in 950 mL of distilled water, and then distilled water was added into the obtained solution to make up to 1 L; the pH was adjusted to a desired value with dilute sulfuric acid and a sodium hydroxide solution; and the product was sterilized with high-pressure steam at 121° C. for 20 min.

3.3 Effect of Temperature on Growth of strain XHZG06-95A3 and Ability Thereof to Increase Ambient pH

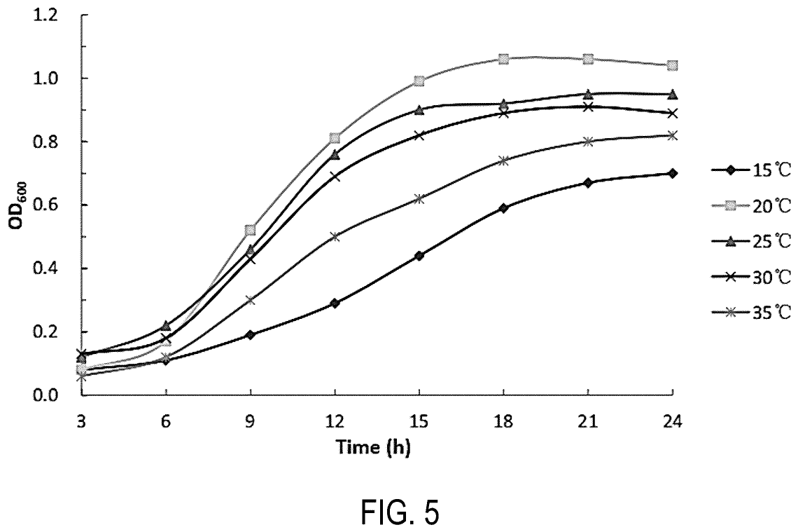

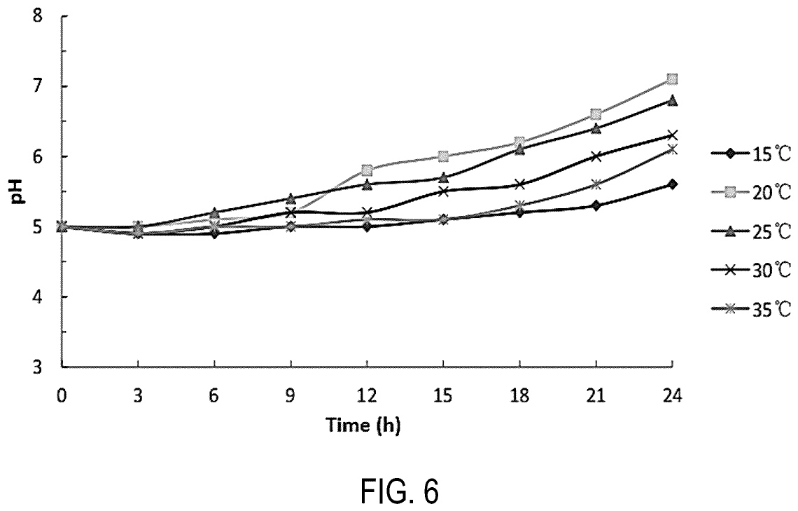

The strain XHZG06-95A3 was inoculated into an LB solid medium, and cultured at 25° C. until single colonies grew out; the single colonies were picked and inoculated into a culture medium with pH of 5.0; the culture temperature was set to 15° C., 20° C., 25° C., 30° C., and 35° C., respectively; shake culture was carried out at 180 r/min and each of the set culture temperatures, and samples were taken at regular intervals; and the absorbance values OD₆₀₀ of the culture medium at 600 nm and the pH values thereof were detected. Table 3, Table 4, FIG. 5 and FIG. 6 show the changes in OD₆₀₀ and pH values of the culture medium over time at different temperatures.

TABLE 3
Effect of temperature on growth of strain XHZG06-95A3

| | OD₆₀₀ values of a culture medium after different time | | | | | | | |
|---|---|---|---|---|---|---|---|---|
| Temperature | 3 h | 6 h | 9 h | 12 h | 15 h | 18 h | 21 h | 24 h |
| 15° C. | 0.08 | 0.11 | 0.19 | 0.29 | 0.44 | 0.59 | 0.67 | 0.70 |
| 20° C. | 0.08 | 0.17 | 0.52 | 0.81 | 0.99 | 1.06 | 1.06 | 1.04 |
| 25° C. | 0.12 | 0.22 | 0.46 | 0.76 | 0.90 | 0.92 | 0.95 | 0.95 |
| 30° C. | 0.13 | 0.18 | 0.43 | 0.69 | 0.82 | 0.89 | 0.91 | 0.89 |
| 35° C. | 0.06 | 0.12 | 0.30 | 0.50 | 0.62 | 0.74 | 0.80 | 0.82 |

TABLE 4
Effect of temperature on pH raising effect of strain XHZG06-95A3

| | pH values of a culture medium after different time | | | | | | | | |
|---|---|---|---|---|---|---|---|---|---|
| Temperature | 0 h | 3 h | 6 h | 9 h | 12 h | 15 h | 18 h | 21 h | 24 h |
| 15° C. | 5.0 | 4.9 | 4.9 | 5.0 | 5.0 | 5.1 | 5.2 | 5.3 | 5.6 |
| 20° C. | 5.0 | 5.0 | 5.1 | 5.2 | 5.8 | 6.0 | 6.2 | 6.6 | 7.1 |
| 25° C. | 5.0 | 5.0 | 5.2 | 5.4 | 5.6 | 5.7 | 6.1 | 6.4 | 6.8 |
| 30° C. | 5.0 | 4.9 | 5.0 | 5.2 | 5.2 | 5.5 | 5.6 | 6.0 | 6.3 |
| 35° C. | 5.0 | 4.9 | 5.0 | 5.0 | 5.1 | 5.1 | 5.3 | 5.6 | 6.1 |

Analysis and conclusion of the test results: It can be seen from Table 3, Table 4, FIG. 5 and FIG. 6 that when the temperature is 15-35° C., the strain XHZG06-95A3 can grow well and increase the pH of the culture medium, so that the strain can function in most ambient temperatures.

Example 4: Effect of temperature on capacity of dissolving P components by strain XHZG06-95A3

In order to explore the effect of temperature on capacity of dissolving P components by a strain XHZG06-95A3, this example used a phosphate-dissolving culture medium containing phosphate rock powder to test the capacity of dissolving P components by the strain XHZG06-95A3 at different temperatures. The specific process and test results are as follows:

4.1 Preparation of medium

An LB solid medium and a phosphate-dissolving culture medium were prepared according to the following formulas.

(1) LB solid medium:

10 g of peptone, 5 g of yeast extract, 10 g of NaCl and 15 g of agar were dissolved in 950 mL of distilled water, and then distilled water was added into the obtained solution to make up to 1 L; the pH was adjusted to 6.0 with dilute sulfuric acid and a sodium hydroxide solution; and the product was sterilized with high-pressure steam at 121° C. for 20 min.

(2) Phosphate-dissolving culture medium 10 g/L yeast extract, 15 g/L glucose, 20 g/L peptone, 0.5 g/L Mg₂SO₄·7H₂O and 0.1 g/L KCl were dissolved in 950 mL of distilled water, 5 g/L of phosphate rock powder was added and evenly dispersed, and then distilled water was added into the obtained solution to make up to 1 L; the pH was adjusted to a desired value with dilute sulfuric acid and a sodium hydroxide solution; and the product was sterilized with high-pressure steam at 121° C. for 20 min.

### 4.2 Effect of Temperature on Capacity of Dissolving P Components by Strain XHZG06-95A3

Experimental group (with inoculation of strain): The strain XHZG06-95A3 was inoculated into an LB solid medium, and cultured at 25° C. until single colonies grew out; the single colonies were picked and inoculated into a phosphate-dissolving culture medium with pH of 6.0; the culture temperature was set to 15° C., 20° C., 25° C., 30° C., and 35° C., respectively; shake culture was carried out at 180 r/min and each of the set culture temperatures, and samples were taken at regular intervals; after centrifugation, the supernatant was taken; and the soluble phosphorus concentration in the supernatant was detected by ammonium molybdate colorimetry.

Control group (without inoculation of strain): A phosphate-dissolving culture medium with pH of 6.0 was taken; the temperature was set to 15° C., 20° C., 25° C., 30° C., and 35° C., respectively; shake culture was carried out at 180 r/min and each of the set temperatures, and samples were taken at regular intervals; after centrifugation, the supernatant was taken; and the soluble phosphorus concentration in the supernatant was detected by ammonium molybdate colorimetry.

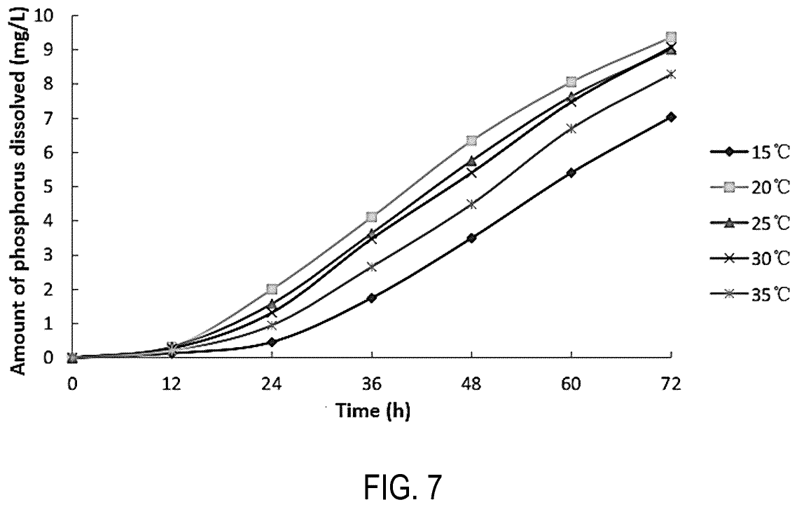

According to the soluble phosphorus concentrations measured in the experimental group and the control group, the amount of phosphorus dissolved was calculated using the following formula: amount of phosphorus dissolved=soluble phosphorus concentration in supernatant of experimental group-soluble phosphorus concentration in supernatant of control group. Table 5 and FIG. 7 show the changes in amount of phosphorus dissolved over time at different temperatures.

TABLE 5
Effect of temperature on capacity of dissolving
P components by strain XHZG06-95A3

| | Amount of phosphorus dissolved after different time (mg/L) | | | | | | |
|---|---|---|---|---|---|---|---|
| Temperature | 0 h | 12 h | 24 h | 36 h | 48 h | 60 h | 72 h |
| 15° C. | 0 | 0.13 | 0.46 | 1.75 | 3.50 | 5.41 | 7.04 |
| 20° C. | 0 | 0.31 | 2.00 | 4.11 | 6.34 | 8.06 | 9.37 |
| 25° C. | 0 | 0.32 | 1.58 | 3.64 | 5.76 | 7.64 | 9.01 |
| 30° C. | 0 | 0.27 | 1.32 | 3.48 | 5.41 | 7.48 | 9.08 |
| 35° C. | 0 | 0.21 | 0.95 | 2.66 | 4.49 | 6.70 | 8.29 |

Analysis and conclusion of the test results: It can be seen from Table 5 and FIG. 7 that the strain XHZG06-95A3 has a good adaptability to temperature, and can effectively play a role in dissolving P components at the temperature of 15-35° C., where its efficiency of dissolving P components is relatively high at the temperature of 20-30° C.

### EXAMPLE 5

### Effect of Initial pH on Capacity of Dissolving P Components by Strain XHZG06-95A3

In order to explore the effect of initial pH on capacity of dissolving P components by a strain XHZG06-95A3, this example used a phosphate-dissolving culture medium containing phosphate rock powder to test the capacity of dissolving P components by the strain XHZG06-95A3 at different initial pH values. The specific process and test results are as follows:

### 5.1 Preparation of Medium

An LB solid medium and a phosphate-dissolving culture medium were prepared according to the following formulas.

### (1) LB Solid Medium:

10 g of peptone, 5 g of yeast extract, 10 g of NaCl and 15 g of agar were dissolved in 950 mL of distilled water, and then distilled water was added into the obtained solution to make up to 1 L;

the pH was adjusted to 6.0 with dilute sulfuric acid and a sodium hydroxide solution; and the product was sterilized with high-pressure steam at 121° C. for 20 min.

### (2) Phosphate-Dissolving Culture Medium

10 g/L yeast extract, 15 g/L glucose, 20 g/L peptone, 0.5 g/L Mg₂SO₄·7H₂O and 0.1 g/L KCI were dissolved in 950 mL of distilled water, 5 g/L of phosphate rock powder was added and evenly dispersed, and then distilled water was added into the obtained solution to make up to 1 L; the pH was adjusted to a desired value with dilute sulfuric acid and a sodium hydroxide solution; and the product was sterilized with high-pressure steam at 121° C. for 20 min.

### 5.2 Effect of Initial pH on Capacity of Dissolving P Components by Strain XHZG06-95A3

Experimental group (with inoculation of strain): The strain XHZG06-95A3 was inoculated into an LB solid medium, and cultured at 25° C. until single colonies grew out; the single colonies were picked and respectively inoculated into phosphate-dissolving culture media with pH of 4.0, 5.0, 6.0, and 7.0; shake culture was carried out at 25° C. and 180 r/min, and samples were taken at regular intervals; after centrifugation, the supernatant was taken; and the soluble phosphorus concentration in the supernatant was detected by ammonium molybdate colorimetry. A phosphate-dissolving culture medium without inoculation of strain XHZG06-95A3 was used as a control.

Control group (without inoculation of strain): Phosphate-dissolving culture media with pH of 4.0, 5.0, 6.0, and 7.0 were taken, respectively; shake culture was carried out at 25° C. and 180 r/min, and samples were taken at regular intervals; after centrifugation, the supernatant was taken; and the soluble phosphorus concentration in the supernatant was detected by ammonium molybdate colorimetry.

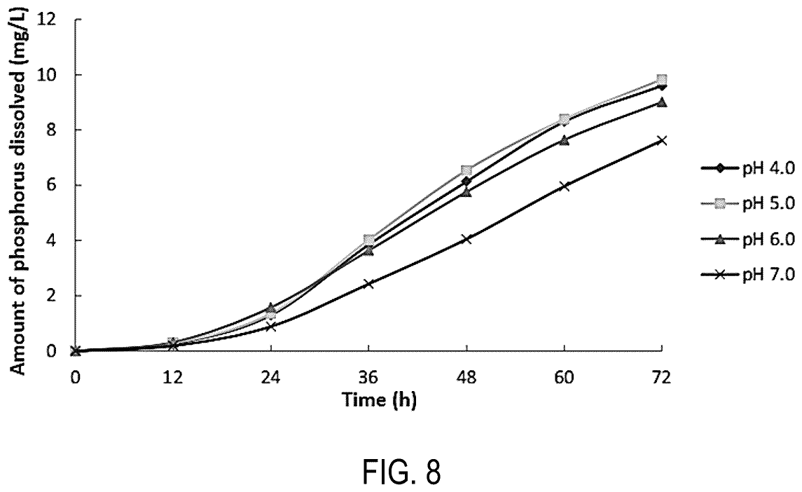

According to the soluble phosphorus concentrations measured in the experimental group and the control group, the amount of phosphorus dissolved was calculated using the following formula: amount of phosphorus dissolved=soluble phosphorus concentration in supernatant of experimental group-soluble phosphorus concentration in supernatant of control group. Table 6 and FIG. 8 show the changes in amount of phosphorus dissolved over time at different initial pH values.

TABLE 6
Effect of initial pH on capacity of dissolving
P components by strain XHZG06-95A3

| Initial | Amount of phosphorus dissolved after different time (mg/L) | | | | | | |
|---|---|---|---|---|---|---|---|
| pH | 0 h | 12 h | 24 h | 36 h | 48 h | 60 h | 72 h |
| pH 4.0 | 0 | 0.26 | 1.30 | 3.85 | 6.13 | 8.30 | 9.60 |
| pH 5.0 | 0 | 0.30 | 1.36 | 4.02 | 6.54 | 8.39 | 9.83 |
| pH 6.0 | 0 | 0.32 | 1.58 | 3.64 | 5.76 | 7.64 | 9.01 |
| pH 7.0 | 0 | 0.19 | 0.89 | 2.43 | 4.05 | 5.96 | 7.62 |

Analysis and conclusion of the test results: It can be seen from Table 6 and FIG. 8 that the strain XHZG06-95A3 has high tolerance to acidic environments and can effectively play a role in dissolving P components when the ambient pH is 4.0-7.0.

### EXAMPLE 6

### Improvement Effect of Strain XHZG06-95A3 on Acidified Soil

In order to explore the improvement effect of a strain XHZG06-95A3 on acidified soil, this example tested the changes in the pH of the soil and the available phosphorus content therein after the application of the strain. The specific process and test results are as follows:

### 6.1 Soil Source

Soil samples were taken from paddy soil after multiple rounds of planting, with pH of 4.9 (soil pH was detected by a water-immersed glass electrode detection method) and available phosphorus content of 22.56 mg/kg (available phosphorus content in the soil was detected by an Olsen method).

### 6.2 Preparation of Medium

An LB solid medium and a culture medium were prepared according to the following formulas.

(1) LB solid Medium:

10 g of peptone, 5 g of yeast extract, 10 g of NaCl and 15 g of agar were dissolved in 950 mL of distilled water, and then distilled water was added into the obtained solution to make up to 1 L; the pH was adjusted to 6.0 with dilute sulfuric acid and a sodium hydroxide solution; and the product was sterilized with high-pressure steam at 121° C. for 20 min.

### (2) Expansion Culture Medium:

10 g/L yeast extract, 15 g/L glucose, 20 g/L peptone, 0.5 g/L K₂HPO₄, 5 g/L (NH₄)₂HPO₄, 0.5 g/L Mg₂SO₄·7H₂O and 0.1 g/L KCI were dissolved in 950 mL of distilled water, and then distilled water was added into the obtained solution to make up to 1 L; the pH was adjusted to 6.0 with dilute sulfuric acid and a sodium hydroxide solution; and the product was sterilized with high-pressure steam at 121° C. for 20 min.

### 6.3 Expanded Culture of Strain XHZG06-95A3

The strain XHZG06-95A3 was inoculated into an LB solid medium, and cultured at 25° C. until single colonies grew out; the single colonies were picked and inoculated into an expansion culture medium; and shake culture was carried out at 20° C. and 180 r/min for 12 h to obtain an XHZG06-95A3 bacterial solution.

### 6.4 Experiment on Improving Acidified Soil

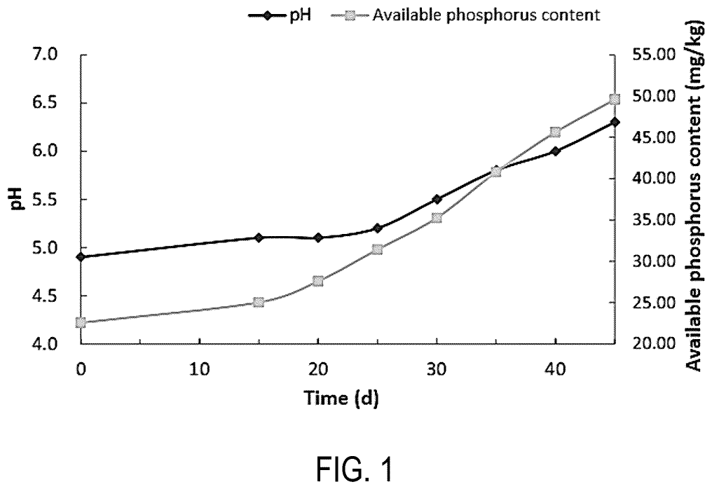

A soil sample was filled into a flower pot with an inner bottom diameter of 21 cm, an inner mouth diameter of 30 cm and a height of 25 cm at an amount of 8 kg/pot. The XHZG06-95A3 bacterial solution was sprayed into the flower pot filled with the soil sample at a spray amount of 8 mL/pot, and then the soil sample was stirred evenly. Samples were taken at regular intervals for detection of pH values and available phosphorus contents of the soil. Table 7 and FIG. 1 show the changes in soil pH and available phosphorus content over time.

TABLE 7
Improvement effect of strain XHZG06-95A3 on acidified soil

| Time (d) | 0 | 15 | 20 | 25 | 30 | 35 | 40 | 45 |
|---|---|---|---|---|---|---|---|---|
| pH value | 4.9 | 5.1 | 5.1 | 5.2 | 5.5 | 5.8 | 6.0 | 6.3 |
| Available | 22.56 | 25.02 | 27.58 | 31.42 | 35.25 | 40.80 | 45.62 | 49.58 |
| phosphorus | | | | | | | | |
| content | | | | | | | | |
| (mg/kg) | | | | | | | | |

Analysis and conclusion of the test results: It can be seen from Table 7 and FIG. 1 that the strain XHZG06-95A3 can effectively increase the pH of the soil and increase the available phosphorus content in the soil after being applied to the acidified soil, thus having a good improvement effect on the acidified soil.

### EXAMPLE 7

### P Components Dissolving Effect of Mussel Shell Powder in Second Fermentation

### 7.1 Preparation of Medium

An LB solid medium and a fermentation culture medium were prepared according to the following formulas.

### (1) LB Solid Medium:

10 g of peptone, 5 g of yeast extract, 10 g of NaCl and 15 g of agar were dissolved in 950 mL of distilled water, and then distilled water was added into the obtained solution to make up to 1 L; the pH was adjusted to 6.0 with dilute sulfuric acid and a sodium hydroxide solution; and the product was sterilized with high-pressure steam at 121° C. for 20 min.

### (2) Fermentation Culture Medium:

10 g/L yeast extract, 15 g/L glucose, 20 g/L peptone, 0.5 g/L K₂HPO₄, 5 g/L (NH₄)₂HPO₄, 0.5 g/L Mg₂SO₄·7H₂O and 0.1 g/L KCl were dissolved in 950 mL of distilled water, and then distilled water was added into the obtained solution to make up to 1 L; the pH was adjusted to 6.5 with dilute sulfuric acid and a sodium hydroxide solution; and the product was sterilized with high-pressure steam at 121° C. for 20 min.

### 7.2 Pretreatment of Mussel Shell Powder

Mussel shell powder with a particle size of 5-10 μm was placed in a muffle furnace and calcined at 600° C. for 3 h to obtain calcined mussel shell powder.

### 7.3 First Fermentation

The strain XHZG06-95A3 was inoculated into an LB solid medium, and cultured at 25° C. until single colonies grew out; the single colonies were picked and inoculated into a fermentation culture medium; and shake culture was carried out at 25° C. and 180 r/min until OD₆₀₀ was 0.8 to obtain a primary fermentation broth.

### 7.4 Second Fermentation

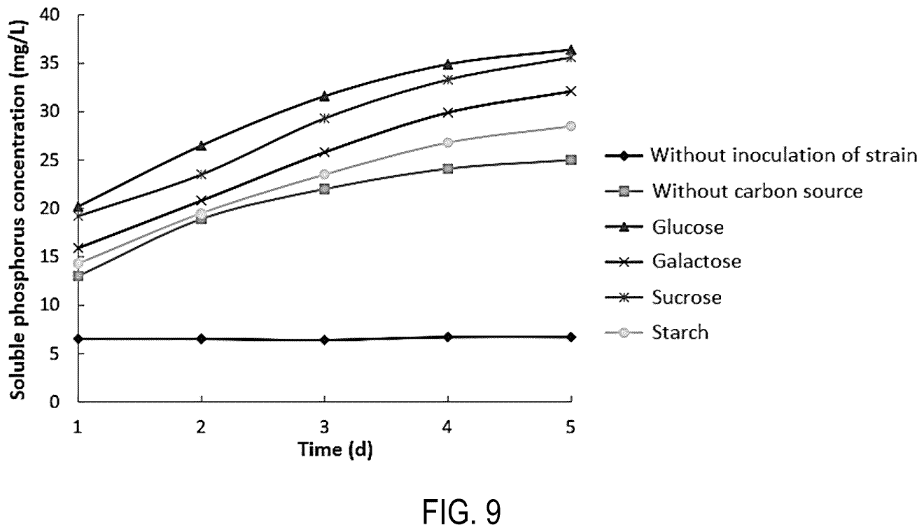

The calcined mussel shell powder was added to the first fermentation broth at an amount of 50 g/L, and then glucose, galactose, sucrose and starch were respectively added as a carbon source, where an adding amount of the carbon source was 5 g/L, with no carbon source added as a control. The obtained mixture was stirred at a speed of 40 r/min at 25° C., and the available phosphorus concentration in the fermentation broth was detected by ammonium molybdate colorimetry at regular intervals. The results are shown in FIG. 9.

A blank control without inoculation of strain XHZG06-95A3 was set: The calcined mussel shell powder was added to the fermentation culture medium at an amount of 50 g/L; the obtained mixture was stirred at a speed of 100 r/min at 25° C.; samples were taken at regular intervals; and after centrifugation, the soluble phosphorus concentration in the supernatant was detected by ammonium molybdate colorimetry. The results are shown in FIG. 9.

Analysis and conclusion of the test results: It can be seen from FIG. 9 that compared with the blank control without using the strain XHZG06-95A3, adding the calcined mussel shell powder into the primary fermentation broth prepared by fermentation of the strain XHZG06-95A3 for the second fermentation can significantly increase the soluble phosphorus concentration in the secondary fermentation broth, which indicates that the strain XHZG06-95A3 can transform the insoluble phosphorus in the mussel shell powder into soluble phosphorus; and furthermore, by adding glucose, galactose, sucrose or starch as a carbon source before the second fermentation, the speed at which the strain XHZG06-95A3 transforms insoluble phosphorus the mussel shell powder into soluble phosphorus can be accelerated. The further preferred carbon source includes glucose and sucrose.

### EXAMPLE 8

### Preparation of Acidified Soil Improvement Agent (Sample #1)

### 8.1 Preparation of Medium

An LB solid medium and a fermentation culture medium were prepared according to the following formulas.

### (1) LB Solid Medium:

10 g of peptone, 5 g of yeast extract, 10 g of NaCl and 15 g of agar were dissolved in 950 mL of distilled water, and then distilled water was added into the obtained solution to make up to 1 L; the pH was adjusted to 6.5 with dilute sulfuric acid and a sodium hydroxide solution; and the product was sterilized with high-pressure steam at 121° C. for 20 min.

### (2) Fermentation Culture Medium:

10 g/L yeast extract, 15 g/L glucose, 20 g/L peptone, 0.5 g/L K₂HPO₄, 5 g/L (NH₄)₂HPO₄, 0.5 g/L Mg₂SO₄.7H₂O and 0.1 g/L KCl were dissolved in 950 mL of distilled water, and then distilled water was added into the obtained solution to make up to 1 L; the pH was adjusted to 6.0 with dilute sulfuric acid and a sodium hydroxide solution; and the product was sterilized with high-pressure steam at 121° C. for 20 min.

### 8.2 Pretreatment of Mussel Shell Powder

Mussel shell powder with a particle size of 5-10 μm was placed in a muffle furnace and calcined at 600° C. for 3 h to obtain calcined mussel shell powder.

### 8.3 First Fermentation

The strain XHZG06-95A3 was inoculated into an LB solid medium, and cultured at 25° C. until single colonies grew out; the single colonies were picked and inoculated into a fermentation culture medium; and shake culture was carried out at 25° C. and 180 r/min until OD600 was 0.8 to obtain a primary fermentation broth.

### 8.4 Second Fermentation

The calcined mussel shell powder was added to the first fermentation broth at an amount of 50 g/L, and then glucose was respectively added at an amount of 5 g/L; and the obtained mixture was stirred at a speed of 40 r/min at 25° C. for 3 days to obtain a second fermentation broth. The secondary fermentation broth was concentrated under reduced pressure and then freeze-dried to obtain an acidified soil improvement agent.

### EXAMPLE 9

### Preparation of Acidified Soil Improvement Agent (Sample #2)

### 9.1 Preparation of Medium

An LB solid medium and a fermentation culture medium were prepared according to the following formulas.

### (1) LB Solid Medium:

10 g of peptone, 5 g of yeast extract, 10 g of NaCl and 15 g of agar were dissolved in 950 mL of distilled water, and then distilled water was added into the obtained solution to make up to 1 L; the pH was adjusted to 6.5 with dilute sulfuric acid and a sodium hydroxide solution; and the product was sterilized with high-pressure steam at 121° C. for 20 min.

### (2) Fermentation Culture Medium:

10 g/L yeast extract, 15 g/L glucose, 20 g/L peptone, 0.5 g/L K₂HPO₄, 5 g/L (NH₄)₂HPO₄, 0.5 g/L Mg₂SO_{4.7}H₂O and 0.1 g/L KCl were dissolved in 950 mL of distilled water, and then distilled water was added into the obtained solution to make up to 1 L; the pH was adjusted to a desired value with dilute sulfuric acid and a sodium hydroxide solution; and the product was sterilized with high-pressure steam at 121° C. for 20 min.

### 9.2 First Fermentation

The strain XHZG06-95A3 was inoculated into an LB solid medium, and cultured at 25° C. until single colonies grew out; the single colonies were picked and inoculated into a fermentation culture medium; and shake culture was carried out at 25° C. and 180 r/min until OD600 was 0.8 to obtain a primary fermentation broth.

### 9.3 Second Fermentation

Mussel shell powder with a particle size of 5-10 μm was added to the first fermentation broth at an amount of 50 g/L, and then glucose was respectively added at an amount of 5 g/L; and the obtained mixture was stirred at a speed of 40 r/min at 25° C. for 3 days to obtain a second fermentation broth. The secondary fermentation broth was concentrated under reduced pressure and then freeze-dried to obtain an acidified soil improvement agent.

### EXAMPLE 10

### Preparation of Acidified Soil Improvement Agent (Sample #3)

### 10.1 Preparation of Medium

An LB solid medium and a fermentation culture medium were prepared according to the following formulas.

### (1) LB Solid Medium:

10 g of peptone, 5 g of yeast extract, 10 g of NaCl and 15 g of agar were dissolved in 950 mL of distilled water, and then distilled water was added into the obtained solution to make up to 1 L;

the pH was adjusted to 6.0 with dilute sulfuric acid and a sodium hydroxide solution; and the product was sterilized with high-pressure steam at 121° C. for 20 min.

### (2) Fermentation Culture Medium:

5 g/L yeast extract, 20 g/L glucose, 20 g/L peptone, 1 g/L K₂HPO₄, 3 g/L (NH₄)₂HPO₄, 0.5 g/L Mg₂SO₄·7H₂O and 0.3 g/L KCl were dissolved in 950 mL of distilled water, and then distilled water was added into the obtained solution to make up to 1 L; the pH was adjusted to 6.0 with dilute sulfuric acid and a sodium hydroxide solution; and the product was sterilized with high-pressure steam at 121° C. for 20 min.

### 10.2 Pretreatment of Mussel Shell Powder

Mussel shell powder with a particle size of 5-10 μm was placed in a muffle furnace and calcined at 550° C. for 4 h to obtain calcined mussel shell powder.

### 10.3 First Fermentation

The strain XHZG06-95A3 was inoculated into an LB solid medium, and cultured at 25° C. until single colonies grew out; the single colonies were picked and inoculated into a fermentation culture medium; and shake culture was carried out at 25° C. and 180 r/min until OD₆₀₀ was 1.0 to obtain a primary fermentation broth.

### 10.4 Second Fermentation

The calcined mussel shell powder was added to the first fermentation broth at an amount of 20 g/L, and then glucose was respectively added at an 20 g/L; and the obtained mixture was stirred at a speed of 40 r/min at 25° C. for 1 day to obtain a second fermentation broth. The secondary fermentation broth was concentrated under reduced pressure and then freeze-dried to obtain an acidified soil improvement agent.

### EXAMPLE 11

### Preparation of Acidified Soil Improvement Agent (Sample #4)

### 11.1 Preparation of Medium

An LB solid medium and a fermentation culture medium were prepared according to the following formulas.

### (1) LB Solid Medium:

10 g of peptone, 5 g of yeast extract, 10 g of NaCl and 15 g of agar were dissolved in 950 mL of distilled water, and then distilled water was added into the obtained solution to make up to 1 L;

the pH was adjusted to 7.0 with dilute sulfuric acid and a sodium hydroxide solution; and the product was sterilized with high-pressure steam at 121° C. for 20 min.

### (2) Fermentation Culture Medium:

10 g/L yeast extract, 20 g/L glucose, 10 g/L peptone, 0.5 g/L K₂HPO₄, 6 g/L (NH₄)₂HPO₄ and 1 g/L Mg₂SO₄·7H₂O were dissolved in 950 mL of distilled water, and then distilled water was added into the obtained solution to make up to 1 L; the pH was adjusted to a desired value with dilute sulfuric acid and a sodium hydroxide solution; and the product was sterilized with high-pressure steam at 121° C. for 20 min.

### 11.2 Pretreatment of Mussel Shell Powder

Mussel shell powder with a particle size of 5-10 μm was placed in a muffle furnace and calcined at 650° C. for 3 h to obtain calcined mussel shell powder.

### 11.3 First Fermentation

The strain XHZG06-95A3 was inoculated into an LB solid medium, and cultured at 25° C. until single colonies grew out; the single colonies were picked and inoculated into a fermentation culture medium; and shake culture was carried out at 25° C. and 180 r/min until OD600 was 0.7 to obtain a primary fermentation broth.

### 11.4 Second Fermentation

The calcined mussel shell powder was added to the first fermentation broth at an amount of 80 g/L, and the obtained mixture was stirred at a speed of 40 r/min at 25° C. for 5 days to obtain a second fermentation broth. The secondary fermentation broth was concentrated under reduced pressure and then freeze-dried to obtain an acidified soil improvement agent.

### EXAMPLE 12

### Remediation Effect of Acidified Soil Improvement Agent on Acidified Soil

### 12.1 Soil Source

Soil samples were taken from paddy soil after multiple rounds of planting, with pH of 4.9 (soil pH was detected by a water-immersed glass electrode detection method) and available phosphorus content of 22.56 mg/kg (available phosphorus content in the soil was detected by an Olsen method).

### 12.2 Acidified Soil Improvement Agent Treatment

Soil samples were filled into flower pots, each of which has an inner bottom diameter of 21 cm, an inner mouth diameter of 30 cm and a height of 25 cm, at an amount of 8 kg/pot. The acidified soil improvement agents (samples #1-#4) prepared in Examples 4-7 were added into the flower pots filled with the soil samples, with each of mussel shell powder and the primary fermentation broth (prepared according to the method in Example 4) of a strain XHZG06-95A3 taken as a control. The addition amounts of the acidified soil improvement agents and the mussel shell powder were 0.3 kg/pot, and the addition amount of the primary fermentation broth was 10 mL/pot.

The soil samples were stirred evenly. After 15 days and 30 days, samples were taken to detect the pH and available phosphorus content of the soil. The results are shown in Table 8.

TABLE 8
Improvement effect of acidified soil

| | |
|---|---|
| 15 d | 30 d |

| | | Available | | Available |
|---|---|---|---|---|
| Acidified soil | | phosphorus | | phosphorus |
| improvement | | content | | content |
| agent | pH | (mg/kg) | pH | (mg/kg) |
| Mussel shell | 5.3 | 23.82 | 5.4 | 24.10 |
| powder | | | | |
| Primary | 5.1 | 27.50 | 5.7 | 39.31 |
| fermentation | | | | |
| broth | | | | |
| Sample #1 | 5.7 | 39.37 | 6.8 | 52.23 |
| Sample #2 | 5.4 | 34.33 | 6.3 | 46.56 |
| Sample #3 | 5.5 | 37.60 | 6.5 | 49.72 |
| Sample #4 | 5.5 | 34.71 | 6.6 | 47.05 |

### Analysis and Conclusion of the Test Results:

(1) From Table 8, it can be seen that the application of the primary fermentation broth of the strain XHZG06-95A3 can increase the pH and available phosphorus content of acidified soil, indicating that the strain has the ability to degrade or neutralize acidic substances in soil, and has the capacity of dissolving P components, and thus it can transform insoluble phosphorus in soil into soluble phosphorus that can be absorbed and utilized by plants.
(2) From Table 8, it can be seen that compared to using the mussel shell powder or strain XHZG06-95A3 alone, the acidified soil improvement bacterium prepared using the same in the 10 present invention can better improve the pH and available phosphorus content of acidified soil.

Unless otherwise defined, all technical and scientific terms used in the present invention have the same meanings as those commonly understood by those skilled in the art to which this disclosure belongs. The raw materials and equipment used in the present invention are, unless otherwise specified, conventional materials and equipment in the art, and can be obtained through conventional commercial channels. The methods used in the present invention are conventional methods in the art, unless otherwise specified.

5 The foregoing descriptions are merely exemplary examples of the present invention, and are not intended to limit the present invention in any way. Any simple modifications, changes or equivalent variations made to the above examples based on the technical essence of the present invention still fall within the protection scope of the technical solutions of the present invention.

1. A strain of Candida XHZG06-95A3, wherein the Candida XHZG06-95A3 belongs to Candida palmioleophila in taxonomy, and has a preservation number of CGMCC No. 29215.

2. A method for preparing an acidified soil improvement agent based on microbial fermentation, comprising the following steps:
(1) inoculating an acidified soil improvement bacterium into a fermentation culture medium for first fermentation to obtain a primary fermentation broth, wherein the acidified soil improvement bacterium is Candida XHZG06-95A3 belonging to Candida palmioleophila in taxonomy, and having a preservation number of CGMCC No. 29215;
(2) adding mussel shell powder to the primary fermentation broth for second fermentation to obtain a secondary fermentation broth; and
(3) separating products from the secondary fermentation broth to obtain an acidified soil improvement agent.

3. The method according to claim 2, wherein, in the step (1), the fermentation culture medium is prepared from components at the following concentrations: 5-10 g/L yeast extract, 10-20 g/L glucose, 10-20 g/L peptone, 0.5-1 g/L K₂HPO₄, 3-6 g/L (NH₄)₂HPO₄, 0.5-1 g/L Mg₂SO₄·7H₂O, and 0-0.3 g/L KCI, with water used as a solvent and pH being 6-7.

4. The method according to claim 2, characterized in that, wherein, in the step (1), the primary fermentation broth has an OD₆₀₀ of 0.7-1.2.

5. The method according to claim 2, wherein, in the step (2), a mass-to-volume ratio of the mussel shell powder to the primary fermentation broth is 2-8 g:100 mL.

6. The method according to claim 2, wherein, in the step (2), a carbon source is added to the primary fermentation broth before the second fermentation is carried out, and the carbon source comprises one or more of glucose, galactose, sucrose, and starch.

7. The method according to claim 2, wherein, in the step (2), before the mussel shell powder is added to the primary fermentation broth, the mussel shell powder is calcined at 550-650° C. for 3-4 h.

8. The method according to claim 2, wherein, in the step (2), the mussel shell powder has a particle size of less than or equal to 10 μm.

9. (canceled)

10. An application of the Candida XHZG06-95A3 according to claim 1 in improving acidified soil.

11. The method according to claim 2, wherein, in the step (3), separating products from the secondary fermentation broth is carried out by concentrating the secondary fermentation broth under reduced pressure and then freeze-drying the secondary fermentation broth.

12. The method according to claim 4, wherein, in the step (2), a mass-to-volume ratio of the mussel shell powder to the primary fermentation broth is 2-8 g:100 mL.

13. The method according to claim 7, wherein, in the step (2), the mussel shell powder has a particle size of less than or equal to 10 μm.

14. An application of an acidified soil improvement agent prepared according to claim 2 in improving acidified soil.

15. An application of an acidified soil improvement agent prepared according to claim 3 in improving acidified soil.

16. An application of an acidified soil improvement agent prepared according to claim 4 in improving acidified soil.

17. An application of an acidified soil improvement agent prepared according to claim 5 in improving acidified soil.

18. An application of an acidified soil improvement agent prepared according to claim 6 in improving acidified soil.

19. An application of an acidified soil improvement agent prepared according to claim 7 in improving acidified soil.

20. An application of an acidified soil improvement agent prepared according to claim 8 in improving acidified soil.

21. An application of an acidified soil improvement agent prepared according to claim 11 in improving acidified soil.
